(19) United States
(12) Patent Application Publication
(10) Pub. No.: US 2026/0157946 A1
(43) Pub. Date: Jun. 11, 2026

(54) BROAD-SPECTRUM, MINERAL, PHOTOPROTECTIVE COMPOSITIONS

(71) Applicant: LAURA C SINGLETON, INGLEWOOD, CA (US)

(72) Inventor: LAURA C SINGLETON, INGLEWOOD, CA (US)

(21) Appl. No.: 19/379,637

(22) Filed: Nov. 4, 2025

Related U.S. Application Data
(63) Continuation of application No. 15/934,312, filed on Mar. 23, 2018, now abandoned.
(63) Continuation of application No. 18/357,695, filed on Jul. 24, 2023, now Pat. No. 12,605,314.

(51) Int. Cl. A61K 8/27; A61K 8/06; A61K 8/25; A61K 8/29; A61K 8/891; A61K 8/96; A61Q 17/04

(52) U.S. Cl. CPC A61K 8/27; A61K 8/064; A61K 8/25; A61K 8/29; A61K 8/891; A61K 8/96; A61Q 17/04; A61K 2800/412

(57) ABSTRACT

Broad-spectrum, stable, water-in-oil emulsion, mineral sunscreen compositions comprising (a) a formulation component comprising (I) a water-in-oil emulsifier component comprising at least one polyglyceryl fatty acid ester compound containing 3-6 glycerin units, (II) two film-forming polymers, e.g., copolymers of dimethicone, (b) a siliceous compound component consisting of two siliceous compounds (I) an amorphous spherical silica component, and (II) a mixture of diatomaceous algae materials comprising materials from at least two of Fragilaria zeilleri, Gomphonema angustatum, Navicula radiosa, and Cyclotella andancensis (c) a sunscreen active ingredient component comprising (I) two photoprotective metal oxides that block, reflect, refract or otherwise attenuate ultraviolet radiation comprising (A) highly porous zinc oxide particles having an average particle size of greater than about 100 nanometers and infiltrated by the formulation component (B) and titanium dioxide particles, wherein the zinc oxide and titanium dioxide particles are evenly dispersed in the compositions.

### RELATED APPLICATIONS/PRIORITY

This patent application is a continuation of and claims priority to presently co-pending U.S. patent application Ser. No. 18/357,695, filed Jul. 24, 2023, entitled “Broad-Spectrum, Mineral, Photoprotective Compositions,” which is a continuation of U.S. patent application Ser. No. 15/934,312, filed Mar. 23, 2018, entitled “Broad-Spectrum, Mineral, Photoprotective Compositions, now abandoned. This application claims the benefit of priority to, and incorporates by reference the entirety of, the above-referenced priority applications.

### FIELD OF THE INVENTION

Topical compositions that provide broad-spectrum (defined below) photoprotection across the ultraviolet radiation spectrum (from 280-400 nm).

### BACKGROUND OF THE INVENTION

The scientific literature and media have raised health concerns (possible carcinogenicity and endocrine disruption) and other concerns (instability) with respect to certain “chemical” sunscreen filters (also known in the art as “organic” sunscreen filters, defined below).

Avobenzone, the most common organic sunscreen in broad-spectrum chemical sunscreen formulations, is known to undergo rapid photochemical degradation when exposed to ultraviolet radiation and is, therefore, considered “unstable.” See, e.g., Kumar P et al., “Patent review on photostability enhancement of avobenzone and its formulations.” Recent Pat Drug Deliv Formul. Vol. 9, No. 2, pp. 121-8 (2015); Afonso, S. et al. “Photodegradation of avobenzone: stabilization effect of antioxidants” J Photochem Photobiol B. Vol. 140, pp. 36-40 (2014); Beasley, D G et al., “Characterization of the UVA protection provided by avobenzone, zinc oxide, and titanium dioxide in broad-spectrum sunscreen products.” Am J Clin Dermatol Vol. 11, No. 6, pp. 413-21 (2010).

Benzophenone-3, also known as oxybenzone, not only absorbs UVB and short UVA rays, but also prevents the degradation of other chemical sunscreens when exposed to UVR. In a podium presentation at the September 2007 Sunscreen Symposium organized by the Florida Chapter of the U.S. Society of Cosmetic Chemists, Craig Bonda reported that oxybenzone quenches avobenzone fluorescence, reducing the flow of singlet excited state energy to the triplet-excited state, and thereby decreasing the potential for chemical reactions that would photodegrade avobenzone.

Health concerns have, however, been raised about the potential adverse health effects from dermal absorption of benzophenone-3. In June 2012, the Office of Environmental Health Hazard Assessment (OEHHA) within the California Environmental Protection Agency added benzophenone-3 to the list of chemicals known to the State of California to cause cancer for purposes of the Safe Drinking Water and Toxic Enforcement Act of 1986 (Proposition 65).

Another commonly used ingredient to stabilize avobenzone is the UV-B organic sunscreen filter, octocrylene. Health concerns have also been raised with respect to octocrylene. In 2010, in an article entitled “Octocrylene, an emerging photoallergen” published in Archives of Dermatology [Vol. 146, No. 7, pp. at pages 753-757 (2010)], Avenel-Audran M et al. reported that octocrylene appears to be a strong allergen leading to contact dermatitis in children and mostly photoallergic contact dermatitis (in particular, in adults with a history of photo allergy from ketoprofen). See also, de Groot, A C and D W Roberts, “Contact and photocontact allergy to octocrylene: a review.” Contact Dermatitis, Vol. 70, pp. 193-204. (2014).

The Environmental Working Group has raised health concerns regarding several organic sunscreen filters. See ewg.org/sunscreen/report/the-trouble-with-sunscreen-chemicals/, citing two publications (among others) by Janjua N R, et al.: Janjua N R, et al. “Systemic absorption of the sunscreens benzophenone-3, octyl-methoxycinnamate, and 3-(4-methylbenzylidene) camphor after whole-body topical application and reproductive hormone levels in humans.” J. Invest. Dermatol. Vol. 123, pp. 57-61 (2004); Janjua N R, et al. “Sunscreens in human plasma and urine after repeated whole-body topical application.” J Eur. Acad. Dermatol. Venereal. Vol. 22, No. 4, pp. 456-461 (2008).

Physical sunscreen filters (most commonly zinc oxide and titanium dioxide), while not raising health concerns of the type associated with organic/chemical sunscreen filters (as discussed above), nonetheless pose challenges both in terms of formulation and aesthetics. Achieving high levels of UVR photoprotection (e.g., SPF of at least 30, which can be described as providing “high protection against sunburn and tanning” under current US Food and Drug Administration regulations) with inorganic sunscreen formulations requires high loadings of physical sunscreen filters. Formulations with high particle loadings can become unstable, including agglomeration and settling of particles. In addition, physical sunscreen products with high SPF (greater than 30; and, especially, greater than 50) may be difficult to apply (spread, rub-in) and/or may be perceived as lacking cosmetic elegance (in terms of feel and/or appearance). Use of zinc oxide, particularly at higher concentrations, is known to leave a white, pasty residue on the skin.

There has been, and remains, a need for sunscreen formulations that do not contain organic sunscreen actives, have an SPF of at least 30, and preferably at least 50, provide broad-spectrum UVR protection, and are cosmetically elegant—not oily, not tacky, leave no visible whitening on Fitzpatrick Skin Type I-IV, and are easily applied to the skin (spread). Those needs are met by the compositions of the present invention.

It is well-known in the art of formulating topical products (for personal care, cosmetic, skin care, and dermatologic applications) that achieving a stable emulsion formulation—one in which (i) a discontinuous inner phase is homogenously dispersed into a continuous external phase, and does not separate into two phases, and (ii) in which particles are and remain dispersed and do not settle—is difficult to accomplish with a single surfactant. Consequently, such formulations typically require at least two surfactants. But it is also known in the dermatologic arts that surfactants can have negative effects—both on the user and on the formulation itself. Many surfactants are known to be irritating. Surfactants can also interact with, and reduce, the activity of certain “anti-aging” ingredients (for example, by denaturing peptides). Moreover, surfactants can interact with film-forming polymers, thereby reducing water-resistant properties, which are highly desirable in sunscreens.

There has been, and remains, a need for emulsion sunscreen formulations that are stable, have a high level of water resistance (preferably 80 minutes in accordance with standards set out in Section 352.76 of Title 21 of the U.S. Code of Federal Regulations). These needs are also met by the compositions of the present invention.

### SUMMARY OF THE INVENTION

Disclosed are broad-spectrum mineral sunscreen compositions in the form of water-in-oil (W/O) emulsions that, upon application to skin classified as Fitzpatrick Skin Types I-IV (as described in Appendix A), leave little or no visible deposition of a white “pasty” residue.

Sunscreen compositions of the present invention (a) provide broad-spectrum protection from ultraviolet radiation (“UVR”) and have a sun protection factor (SPF) of at least 30, preferably at least 50, and (b) are water resistant, preferably for 80 minutes, (c) are not visibly non-whitening on Fitzpatrick Skin Types I-IV and (d) do not contain an organic sunscreen filter, and consist essentially of:
(i) two photoprotective metal oxides that block, reflect, refract or otherwise attenuate ultraviolet radiation: at least one zinc oxide particle, preferably uncoated; at least one titanium dioxide particle, preferably coated; and, optionally, one or more other metal oxides (that block, reflect, refract or otherwise attenuate visible, infrared or ultraviolet radiation);
(ii) two siliceous compounds
(a) an amorphous silica, preferably an amorphous spherical silica, and
(b) a mixture of diatomaceous algae comprising at least two, preferably three, and still more preferably all four of Fragilaria zeilleri, Gomphonema angustatum, Navicula radiosa, and Cyclotella andancensis;
(iii) two film-forming polymers, preferably copolymers of dimethicone and
(a) bis-vinyl dimethicone and
(b) acrylates
(iv) two silicone compounds, one silicone wax, (preferably cetyl dimethicone) and one silicone fluid (preferably selected from the group of dimethicone and phenyl trimethicone).

The inventive broad-spectrum sunscreen W/O emulsions of the present invention preferably contain a single emulsifier that (a) has a hydrophilic-lipophilic balance in the range of 3.0 to 8.0, more preferably 3.0 to 5.0 (b) does not contain polyethylene glycol, and (c) is an ester formed with polyglycerin, preferably containing 3-6 glycerin units. It is surprising and unexpected to achieve a stable emulsion (i.e., one that does not separate into oil and water phases after storage at an elevated temperature of 50° C. for one month or 40° C. for three months) with only a single emulsifier.

Surprisingly and unexpectedly, the synergistic combination of an amorphous spherical silica and a mixture of diatomaceous algae comprising at least two, preferably three, and still more preferably all four of Fragilaria zeilleri, Gomphonema angustatum, Navicula radiosa, and Cyclotella andancensis in further combination with zinc oxide and titanium dioxide provides a sun protection factor of at least 50, with a critical wavelength of greater than about 375, without visible skin whitening on Fitzpatrick Skin Types I-IV.

The compositions of the invention also provide one, preferably several, cosmetic skin benefits including (a) reducing the appearance of fine lines/wrinkles, (b) improving skin barrier function (i.e., by reducing the rate/extent of trans-epidermal water loss), (c) making the skin feel smoother, more supple and softer, (d) creating the appearance of more even skin tone (reducing dyschromia) and/or (e) imparting “glow”/radiance (also described in the art as “brightness”).

### DEFINITIONS

“Organic Sunscreen” means small molecule organic chemical sunscreen molecules (also known in the art as ‘active ingredients”) that absorb UVR, and include sunscreen active ingredients that are generally recognized as safe and effective and are approved for over-the-counter use by the U.S. Food and Drug Administration (“FDA”)—namely, avobenzone; cinoxate; dioxybenzone; ecamsule; homosalate; menthyl anthranilate; octocrylene; octyl methoxycinnamate; octyl salicylate; oxybenzone; p-aminobenzoic acid; padimate o; phenylbenzimidazole sulfonic acid; sulisobenzone; and trolamine salicylate. Organic sunscreens also include active ingredients approved by regulatory agencies outside the United States, but not currently approved by the FDA, including, 4-methylbenzylidene camphor, amiloxate, benzophenone-9, Mexoryl® XL, Neo Heliopan® AP, Parsol® SLX, Tinosorb® A2B, Tinosorb® M, Tinosorb® S, Uvasorb® HEB, Uvinul® A Plus, and Uvinul® T 150.

“Photoprotective metal oxide” means a particle that physically blocks ultraviolet, visible and/or infrared light, and thereby protects the skin, and reduces environmentally-caused damage which can manifest as fine lines, wrinkles, uneven pigmentation (dyschromia), loss of elasticity or firmness, increased dryness, reduced skin moisture, and loss of softness/suppleness.

“Water-in-oil emulsion” or “W/O emulsion” means an internal water phase dispersed in an external non-aqueous phase comprised of hydrophobic materials, including oils, esters, and silicones. W/O emulsions can also be understood to include water-in-silicone emulsions.

“Broad-spectrum” refers to a level of protection from UVR provided by wearing a sunscreen that has a minimum critical wavelength of 370 nm and a sun protection factor (SPF) value of 15 or higher.

“Critical wavelength” is the wavelength for which the section under the integrated optical density curve starting at 290 nm is equal to 90 percent of the integrated section between 290 nm to 400 nm. Standards and test methods for determining “broad-spectrum” protection from UVR are set out in the final Sunscreen Monograph promulgated by the FDA on Jun. 17, 2011. See “Labeling and Effectiveness Testing; Sunscreen Drug Products for Over-the-Counter Human Use” published at Volume 76 of the Federal Register starting at page 35620, the disclosure of which is incorporated herein by reference.

Hydrophilic-lipophilic balance (“HLB”) is a measure of the degree to which a surfactant is hydrophilic or lipophilic. HLB may be calculated in accordance with the methods published by Griffin in the Journal of the Society of Cosmetic Chemists: “Classification of Surface-Active Agents by ‘HLB”, J. Soc. Cosm. Chem., Vol. 1, No. 5, pp. 311-26; “Calculation of HLB Values of Non-Ionic Surfactants” J. Soc. Cosm. Chem., Vol. 5, No. 4, pp. 249-56.

“One or more” means at least one and thus includes individual components as well as mixtures/combinations.

Compositions of the present invention consist essentially of, and consist of, the ingredients described hereinbelow, as well as additional (optional) ingredients known to the skilled artisan to provide features or benefits useful in providing protection from UVR (as well as UVR and infrared radiation) and also providing cosmetic/aesthetic benefits that include, but are not limited to, reducing the appearance of fine lines/wrinkles, improving skin barrier function (by reducing the rate/extent of trans-epidermal water loss), making the skin feel smoother/more supple/softer, creating the appearance of more even skin tone (reducing dyschromia) and/or “slow”/radiance (also described in the art as “brightness”).

A basic and novel characteristic of the inventive broad-spectrum photoprotective compositions of the present invention is the absence of organic sunscreens. Accordingly, in describing and claiming such compositions as “consisting essentially of (a) two photoprotective metal oxides that block, reflect, refract or otherwise attenuate ultraviolet radiation . . . ” it is meant that: zinc oxide (preferably uncoated) and titanium dioxide (preferably coated) are both essential required component ingredients; other metal oxides that block, reflect, refract or otherwise attenuate visible, infrared or ultraviolet radiation, including cerium oxide, aluminum oxide, and iron oxides, may be component ingredients; but organic sunscreens are not component ingredients.

Numbers used in describing quantities of ingredients and/or reaction conditions are to be understood as being modified in all instances by the term “about.”

Unless otherwise indicated, percentages, parts and ratios are to be understood as based upon the total weight of the composition.

Numerical ranges are meant to include numbers within the recited range and combinations of subranges between the given ranges. For example, a range from 1-5 includes 1, 2, 3, 4, and 5, as well as subranges such as 2-5, 3-5, 2-3, 2-4, 1-4, etc.

“At least one” means one or more and also includes individual components as well as mixtures/combinations.

“Cosmetically acceptable” (for example, “cosmetically acceptable carrier”) means compatible with a keratinous substrate.

The disclosures of U.S.-granted patents and US pre-grant patent publications are incorporated, in pertinent part, by reference.

### Photoprotective Metal Oxide Particles

Broad-spectrum photoprotective compositions of the present invention contain at least two photoprotective metal oxides—zinc oxide (ZnO) and titanium dioxide (TiO₂).

TiO₂ can be amorphous or crystalline in the rutile and/or anatase form. Preferably 95% or more of the TiO₂ is in the rutile form.

ZnO and TiO₂ can be, and in certain preferred embodiments, are coated. Coating is done by methods known in the art. By way of non-limiting example, starting with an aqueous pigment particle suspension, metal salts are added in dissolved form as so-called precursor compounds. Alkaline or acidic substances are then used to set the pH value of the suspension in such a way that the precursor compounds are precipitated in the form of oxides, hydroxides, etc. Methods for modifying and hydrophobizing the surface of TiO₂ and ZnO are further disclosed, for example, in U.S. Pat. Nos. 5,565,591 and 5,486,631, both of which are now expired.

Preferred, but non-limiting examples of materials that can be used to coat ZnO and TiO₂ are silica, alumina, aluminum hydroxide, aluminum stearate, triethoxycaprylsilane, stearic acid, caprylic/capric triglyceride, lecithin, and methicone.

Silica is the inorganic oxide that conforms to the formula SiO₂.

Alumina is an inorganic compound that conforms to the structure Al₂O₃.

Aluminum hydroxide is an inorganic compound that conforms to the structure Al(OH)₃·xH₂O.

Aluminum stearate is the aluminum salt of stearic acid that conforms to the formula: CH₃(CH₂)₁₆COOAl(OH))₂.

Triethoxycaprylylsilane is a siloxane ether that conforms to the formula: (CH₃CH₂O)₃Si(CH₂)₇CH₃. Stearic acid is a fatty acid that conforms to the structure. (CH₃CH₂)₁₆COOH.

Caprylic/Capric triglyceride is the mixed triester of glycerin and caprylic and capric acids.

Methicone is a linear monomethyl polysiloxane that conforms generally to the formula:

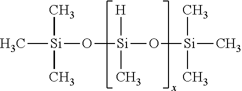

Photoprotective metal oxides may also be a mixture of C₁₂ to C₃₀ fatty alcohols and C₆ to C₁₂ aliphatic acids, as described in U.S. Pat. No. 9,517,190.

TiO₂ useful in W/O emulsion compositions of the present invention may be commercially available in a mixture that is a “binary combination”—namely, TiO₂ and a second ingredient selected from the group of: dimethicone; isopropyl titanium triisostearate; methicone; polymethyl methacrylate; polyphosphorylcholine glycol acrylate; silica; simethicone; stearic acid; and triethoxycaprylylsilane.

TiO₂ useful in broad-spectrum sunscreen compositions of the present invention may also be part of a tripartite combination (i.e., TiO₂ and a second ingredient, and a third ingredient).

In certain embodiments, TiO₂ and Alumina (as a second ingredient) are combined with a third ingredient selected from the group of: glycerin; jojoba esters; methicone; silica; and stearic acid.

In other embodiments, TiO₂ and aluminum hydroxide (as a second ingredient) are combined with a third ingredient selected from the group of: hydrogen dimethicone; isostearic acid; and stearic acid.

In further embodiments, TiO₂ and silica (as a second ingredient) are combined with a third ingredient selected from the group of: Helianthusannuus (sunflower) seed oil; dimethicone; stearic acid; jojoba esters; lauroyl lysine; sodium polyacrylate; and triethoxycaprylylsilane.

In still further embodiments, TiO₂ and polyhydroxystearic acid (as a second ingredient) are combined with a third ingredient selected from the group of: bisabolol; squalane; and jojoba esters.

TiO₂ and caprylic/capric triglyceride may be combined with alumina, and polyhydroxystearic acid and one of: aluminum stearate; methicone; stearic acid; silica

TiO₂ and caprylic/capric triglyceride may be combined with aluminum hydroxide, polyhydroxystearic acid and/or stearic acid.

TiO₂ may be combined with C₁₂₋₁₅ alkyl benzoate, polyolyhydroxystearic acid, and, optionally, but preferably alumina, in further combination with one of: methicone; cyclomethicone; aluminum stearate; stearic acid, and silica.

TiO₂ may also be combined with C₁₂₋₁₅ alkyl benzoate in further combination with
Argania spinosa kernel oil (and) alumina (and) methicone (and) tocopheryl acetate
(and) polyhydroxystearic acid (and) silica
Polyglyceryl-3 polyricinoleate (and) silica (and) stearic acid (and) aminopropyl-triethoxysilane
Stearic acid (and) aluminum hydroxide (and) polyhydroxystearic acid

TiO₂ may be combined with aluminum hydroxide in further combination with
Acrylates copolymer (and) hydrated silica (and) algin
Butyloctyl salicylate (and) isostearic acid (and) C₁₂₋₁₅ alkyl benzoate (and) stearic acid
C₁₂₋₁₅ alkyl benzoate (and) stearic acid (and) polyhydroxystearic acid
Caprylic/Capric triglyceride (and) stearic acid in further combination with (a) sorbitan olivate or (b) polyhydroxystearic acid
Hydrogen dimethicone
Hydrated silica (and) polyphosphorylcholine glycol acrylate
Stearic acid or isostearic acid
Polydimethylsiloxyethyl hexyl dimethicone (and) PEG-9 polydimethylsiloxyethyl dimethicone
Polyglyceryl-4 isostearate (and) cetyl PEG/PPG-10/1 dimethicone (and) hexyl laurate (and) isostearic acid
Simmondsia chinensis (jojoba) seed oil (and) isostearic (and) polyhydroxystearic acid
Simmondsia chinensis (jojoba) seed oil (and) Polyhydroxystearic acid (and) jojoba esters

Non-limiting examples of “coated” TiO₂ suitable for use in broad-spectrum sunscreen compositions of the present invention include the following:
Sunsil® Tin50 from Sunjin Chemical Co. Ltd.: TiO₂ coated with silica, with a ratio of silica to TiO₂ of about 55:45.
Titanium dioxide (at least 78%; typically about 83%) coated with aluminum hydroxide (about 9%) (and) stearic acid (about 8%), available from Tayca Corp. (Osaka, Japan) under the tradename MT-100TV.
Titanium dioxide (74%) coated with silica (11%), aluminum hydroxide (9%), and alginic acid (5%), available from Tayca Corp. as MT100-AQ.
Titanium dioxide (75-82%) coated with silica (13-20%) available from Merck KgaA/EMD Chemicals (Darmstadt, Germany) under the tradename Eusolex® T-AVO.
SiClone® TD-150 (from Presperse Corp., Somerset, New Jersey) about 40% titanium dioxide with an inner coating of aluminum hydroxide and an outer coating of isostearic acid.

TiO₂ may be used in broad-spectrum sunscreen compositions of the present invention in one of the following combinations:
(a) Boron nitride (and) dimethicone (and) isododecane (and) ethylene/VA copolymer
(b) Butylene glycol (and) caprylyl glycol (and) oleth-10 (and) phenoxyethanol (and) polysorbate 60 (and) silica
(c) Butyloctyl salicylate (and) polyhydroxystearic acid (and) dimethicone (and) hydrogen dimethicone
(d) Caprylic/capric triglyceride (and) stearic acid (and) isostearic acid (and) polyhydroxystearic acid (and) polyglyceryl-3 polyricinoleate (and) lecithin
(e) Cyclomethicone (and) bis-PEG/PPG-14/14 dimethicone (and) aluminum stearate
(f) Cyclopentasiloxane (and) dimethicone (and) PEG-10 dimethicone (and) silica
(g) Cyclopentasiloxane (and) PEG-10 dimethicone (and) methicone
(h) Ethylene/acrylic acid copolymer (and) aluminum stearate
(i) Ethylhexyl palmitate (and) polyhydroxystearic acid (and) silica
(j) Glycerin (and) sodium polyacrylate (and) tetrasodium EDTA (and) silica (and) sodium polyphosphate
(k) Hydrogenated polydecene (and) polyhydroxystearic acid (and) one of: dimethicone; stearic acid; or triethoxycaprylylsilane
(l) Isododecane (and) polyhydroxystearic acid (and) methicone
(m) Isohexadecane (and) triethylhexanoin (and) aluminum stearate (and) polyhydroxystearic acid
(n) Isononyl isononanoate (and) methicone (and) polyhydroxystearic acid
(o) Isopropyl myristate (and) polyhydroxystearic acid (and) silica
(p) Isopropyl titanium triisostearate (and) triethoxysilylethyl polydimethylsiloxyethyl dimethicone
(q) Methyl trimethicone (and) hydrogen dimethicone (and) lauryl PEG-9 polydimethylsiloxyethyl dimethicone
(r) Methyl trimethicone (and) PEG-10 dimethicone (and) methicone
(s) Mica (and) dimethicone (and) isododecane (and) ethylene/VA copolymer (and) stearic acid
(t) Octyldodecyl myristate (and) alumina (and) polyhydroxystearic acid (and) methicone
(u) Phenyl trimethicone (and) hexyl laurate (and) stearic acid (and) polyhydroxystearic acid
(v) Polyglyceryl-2 caprate (and) sucrose stearate (and) Simmondsia chinensis (jojoba) seed oil (and) stearic acid (and) glyceryl caprylate (and) squalane
(w) Simmondsia chinensis (jojoba) seed oil (and) aluminum hydroxide (and) polyhydroxystearic acid (and) one of isostearic acid or jojoba esters
(x) Acrylates copolymer (and) hydrated silica (and) (a) algin (and) aluminum hydroxide or (b) polyphosphorylcholine glycol acrylate
(y) Butyloctyl salicylate (and) aluminum hydroxide (and) isostearic acid (and) C₁₂₋₁₅ alkyl benzoate (and) stearic acid
(z) C₁₂₋₁₅ alkyl benzoate (and) polyglyceryl-2 dipolyhydroxystearate (and) silica (and) dimethicone
(aa) Caprylic/capric triglyceride (and) sorbitan olivate (and) stearic acid (and) aluminum hydroxide
(bb) Caprylyl methicone (and) cyclopentasiloxane (and) C₁₂₋₁₅ alkyl benzoate (and) alumina (and) polyhydroxystearic acid (and) triethoxysilylethyl polydimethylsiloxyethyl hexyl dimethicone (and) PEG-9 polydimethylsiloxyethyl dimethicone
(cc) Hydrated silica (and) hydrogen dimethicone and (a) aluminum hydroxide (or) hydrogen dimethicone
(dd) Isododecane (and) alumina (and) methicone (and) polyhydroxystearic acid
(ee) Isododecane (and) polyglyceryl-4 isostearate (and) cetyl PEG/PPG-10/1 dimethicone (and) hexyl laurate (and) aluminum hydroxide (and) isostearic acid
(ff) Isononyl isononanoate (and) polyhydroxystearic acid (and) hydrogen dimethicone (and) dimethicone

One preferred form of titanium dioxide suitable for use in broad-spectrum sunscreen compositions of the present invention is Tinoply E50C (manufactured by Chemland, Co., Ltd., Seoul, Korea) a mixture of caprylic/capric triglyceride, titanium dioxide, alumina, stearic acid, and polyhydroxystearic acid. More particularly, in Tinoply E50C (a) caprylic/capric triglyceride is present at a concentration of 47.0±2.5%, (b) titanium dioxide is present at a concentration of 39.0±2.5%, (c) alumina is present at a concentration of 6.0±2.5%, (d) stearic acid is present at a concentration 5.0±2.5%, and (e) polyhydroxystearic acid is present at a concentration of 3.0±0.5%.

Another preferred form of titanium dioxide suitable for use in broad-spectrum sunscreen compositions of the present invention is Tinoply E30C—a mixture of caprylic/capric triglyceride, titanium dioxide, silica, polyhydroxystearic acid, and methicone (also manufactured by Chemland, Co., Ltd.) in which (a) caprylic/capric triglyceride is present at a concentration of about 68.2±2.5%, (b) titanium dioxide is present at a concentration of at least about 24%, (c) silica is present at a concentration of 4.5±2.5%, (d) polyhydroxystearic acid is present at a concentration 1.8±0.5%, and (e) methicone at a concentration of 1.5±0.5%.

In certain especially preferred embodiments, the sunscreen does not include alumina.

Uncoated zinc oxide particles that can be used in compositions of the present invention are commercially available from numerous suppliers, including under the tradename Z-Cote® (BASF Care Creations, Florham Park, NJ). Examples of other suitable ZnO are disclosed, for example, in U.S. Pat. No. 8,545,891.

In preferred embodiments, ZnO is non-whitening when applied to the skin and has an average particle size greater than about 100 nm (i.e., “non-nano”). Such materials are sometimes described in trade literature as “transparent”. Historically, non-whitening, non-nano ZnO materials were marketed under the tradename ZinClear™ formerly sold by Antaria Limited (Welshpool, Australia). See US Pre-Grant Patent Application Publications 2010/0310871 and 2010/0316582, both abandoned.

In one particularly preferred embodiment, compositions of the present invention contain ZnO particles having an average particle size of greater than 100 nanometers. One such ZnO is ARGA-SUN ZnO CLR-P from Argan Co. (Northridge, California). According to Technical Data Sheets, ARGA-SUN ZnO CLR-P has a highly porous structure that is “infiltrated” (e.g., filled) with excipient, and an average particle size distribution of less than about 800 nanometers (measured using static laser scattering). Because the particles have a refractive index close to the excipient, a “significant increase in transparency” is achieved.

In preferred embodiments of the present invention, prior to mixing ZnO with TiO₂, ZnO is dispersed in polyhydroxystearic acid (PHSA), a polymer of hydroxystearic acid, which is commercially available under the tradename Dispersun DSP OL 300 from Innospec Performance Chemicals (Salisbury, NC). According to technical literature from Innospec, polyhydroxystearic acid increases UV absorption of sunscreens containing pigments by allowing higher concentrations of pigment to be used. Additionally, inclusion of polyhydroxystearic acid is described by Innospec as improving optical transparency and reducing whitening (when a finished formulation is applied to human skin).

In preferred embodiments, polyhydroxystearic acid is present at a concentration of from about 0.25 to 1.5%, preferably at least about 0.5%.

Different molecular weights of polyhydroxystearic acid may be used in broad-spectrum compositions of the present invention.

Optionally, but in certain preferred embodiments, one or more additional Photoprotective Metal Oxides selected from the group of iron oxides, zirconium oxide, bismuth oxychloride and cerium oxide are incorporated in broad-spectrum sunscreen compositions of the present invention.

In certain embodiments, CeO₂ is present in the broad-spectrum photoprotective compositions of the present invention at a concentration of from 0.1 to 2.0%, preferably about 0.25%.

One preferred form of cerium oxide is ARG-SPHERE NIR-1/15BA000—a combination of poly(methyl methacrylate), also known in the art as PMMA, cerium oxide (CeO₂), and aluminum oxide from Argan Co.

PMMA is a spherical ultra-fine texturizing powder, available in various sizes, used in powders to increase smoothness, fluidity and lubricity. It is a polymer of methyl methacrylate, has an empirical formula C₅H₈O₂)ₓ and conforms to the following structure:

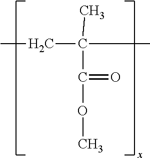

Iron oxides may be present in broad-spectrum photoprotective compositions of the present invention at a concentration of from about 1.0 to 7.0%, preferably from about 1.0 to 3.0%, and still more preferably from about 1.5 to 2.0%.

In tinted formulations (containing three or more iron oxides selected from the group of black iron oxide, red iron oxide, and yellow iron oxide), amorphous spherical silica is preferably present at a concentration of at least 2.0%.

In non-tinted formulations (not containing iron oxides), amorphous spherical silica is preferably present at a concentration of at least 2.5%.

In certain preferred embodiments, a mica, or a mica-like compound that imparts shimmer, glow, or reduces the appearance of skin imperfections, is added to the formulation.

One preferred, but non-limiting example, of a mica-like compound is synthetic fluorphlogopite, a synthetic mineral that conforms generally to the formula: Mg₃K[AlF₂O(SiO₃)₃].

### Siliceous Compounds

Broad-spectrum photoprotective compositions of the present invention contain two siliceous compounds—an amorphous silica and a mixture of diatomaceous algae, each described in detail below.

A first siliceous compound that is an essential ingredient of the broad-spectrum photoprotective compositions of the present invention is amorphous silica, also known in the art as amorphous silicon oxide hydrate. Preferably, the amorphous silica is a spherical, porous powder, still more preferably having a mean particle size ranging from about 6 microns to about 10 microns. One especially preferred amorphous spherical silica is Silisphere LS-8H available from Argan Co.

A second siliceous compound that is an essential ingredient of the broad-spectrum photoprotective compositions of the present invention is a mixture of diatomaceous algae—unicellular, photosynthetic microorganisms having a nano-patterned cell encasement made of amorphous biosilica, also known in the art as a “frustule”, that creates a highly efficient light trapping mechanism. See J. Romann et al. “Wavelength and orientation dependent capture of light by diatom frustule nanostructures” (2015), published online at nature.com/articles/srep17403; X. Chen et al., “Numerical and experimental investigation of light trapping effect of nanostructured diatom frustules” (2015), published online at nature.com/articles/srep11977. See also J. Mishler et al, “Biomimetic Photonic Crystals based on Diatom Algae Frustules” presented at the March 2015 meeting of the American Phytopathological Society, Abstract #A4.004.

Amorphous silica, preferably amorphous spherical silica, is present in broad-spectrum photoprotective compositions of the present invention at a concentration of from about 0.5 to about 5.0%.

Plankton Glass Flower® (INCI Name: “Plankton Extract”), a combination of diatomaceous algal species, is commercially available from Odycea SAS (Lannion, France) and distributed in the United States by Argan Co. (Northridge, California). Technical data sheets and brochures describe Plankton Glass Flower as “algal photonic and porous silica crystals” or, alternatively, “planktonic material” sourced from the lakes in the volcanic region of Auvergne, France—namely, “siliceous fragments of freshwater algae species[,] mainly Fragilaria zeilleri, Gomphonema angustatum, Navicula radiosa, and Cyclotella andancensis”—that contains greater than about 75% silica. Plankton Glass Flower not only provides “UV-visible [light] attenuation due to both reflection and scattering” but also serves as an “oil absorber” and “pollutant scavenger which entraps . . . impurities [at the surface of the skin].”

One, or preferably a mixture of two or more, diatomaceous algal species having photonic and porous silica crystals and a silica content of at least about 75% is/are present in broad-spectrum photoprotective compositions of the present invention at a concentration of from about 0.1 to about 2.0%, preferably from about 0.25 to about 1.0%, and even more preferably at a concentration of at least about 0.5%.

Preferably the one or more diatomaceous algal species having photonic and porous silica crystals and a silica content of at least about 75% are selected from the group consisting of Fragilaria zeilleri, Gomphonema angustatum, Navicula radiosa, and Cyclotella andancensis.

In preferred embodiments of the present invention the ratio of (i) one or more diatomaceous algal species having photonic and porous silica crystals and a silica content of at least about 75% to (ii) amorphous silica, preferably spherical, porous silica, is from about 1:9 to about 1:3.

Total silica content in compositions of the present invention—silica found in (i) one or more diatomaceous algal species having photonic and porous silica crystals and a silica content of at least about 75% and (ii) amorphous silica, preferably amorphous, spherical silica—is preferably from about 1.0 to about 5.0%, more preferably from about 2.0 to about 4.0%.

In certain preferred embodiments that contain CeO₂, the ratio of (1) CeO₂ to (2) Plankton Glass Flower (as described above) to (3) amorphous silica is 1:2:8.

In some especially preferred embodiments of the present invention that are tinted with at least two iron oxides, the ratio of (i) one or more diatomaceous algal species having photonic and porous silica crystals and a silica content of at least about 75% to (ii) amorphous silica, preferably spherical, porous silica, is about 1:4.

In other especially preferred embodiments of the present invention that are not tinted (i.e., do not contain iron oxides), the ratio of (i) one or more diatomaceous algal species having photonic and porous silica crystals and a silica content of at least about 75% to (ii) amorphous silica, preferably spherical, porous silica, is about 1:5.

### SPF Boosters

Broad-spectrum photoprotective compositions of the present invention contain at least one, preferably at least two, and still more preferably three SPF boosters selected from the group of styrene/acrylates copolymer, butyloctyl salicylate, and triethyl citrate (each as described below), and polyhydroxystearic acid (described above).

Styrene/acrylates copolymer is a polymer of styrene and a monomer consisting of acrylic acid, methacrylic acid or one of their simple esters. These hollow spheres are manufactured via emulsion polymerization, and are commercially available from Dow (Midland, Michigan) under the name SUNSPHERES® Powder. A February 2006 Technical Data Sheet (TDS) describes SUNSPHERES® as “rais[ing] UV protection over the whole UVA/UVB spectrum”.

Butyloctyl salicylate (CAS No. 190085-41-7), commercially available as HallBrite® BHB from the Hallstar Company (Chicago, Illinois) and SunSolv® from Innospec Performance Chemicals (Salisbury, North Carolina) is a synthetically produced ester of salicylic acid and a branched C₁₂ alcohol, 2-butyloctanol. Butyloctyl salicylate, and its uses in topical formulations, are described in the following U.S. Pat. Nos. 5,783,173; 5,788,954; 5,849,273; and 6,350,894.

Triethyl citrate is the triester of ethyl alcohol and citric acid that conforms to the formula:

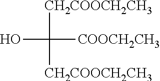

Surprisingly and unexpectedly, broad-spectrum photoprotective compositions of the present invention containing at least one, preferably at least two, and more preferably three of the above SPF boosters provide an SPF of at least 30, and, in preferred embodiments, an SPF of at least 50, without visible whitening on Skin Types I-IV under the Fitzpatrick Skin Type system of skin classification, described in Appendix A.

### Water-In-Oil Emulsifier

In preferred embodiments of the present invention, the inventive broad-spectrum photoprotective composition contains a single emulsifier having a low HLB, by which is meant from about 3.0 to about 8.0, more preferably from about 3.0 to about 5.0, and still more preferably from about 3.5 to about 4.0.

Preferably, the low HLB W/O emulsifier is present at a concentration of from about 2.25% to about 6%, preferably at least about 2.5%, more preferably at least 5%.

One preferred, but non-limiting example, of a low HLB W/0 emulsifier is an ester formed with polyglycerin, preferably containing 3-6 glycerin units. These low HLB W/O emulsifiers are free of polyethylene glycol (i.e., are “PEG-Free”). Non-limiting examples of preferred low HLB W/O ester emulsifiers are polyglyceryl-3 oleate, polyglyceryl-3 ricinoleate, polyglyceryl-3 sesquiisostearate, polyglyceryl-3 polyricinolate, and polyglyceryl-4 oleate.

Polyglyceryl-3 oleate, polyglyceryl-3 ricinoleate, polyglyceryl-3 sesquiisostearate, and polyglyceryl-3 polyricinoleate are all esters formed from polyglycerin-3, a glycerin polymer containing 3 glycerin units.

Polyglyceryl-4 oleate is an ester of oleic acid and polyglycerin-4, a glycerin polymer containing 4 glycerin units.

### Film-Forming Polymers and Silicone Compounds

Siloxanes, also known in the art as organo-substituted polysiloxanes, are linear or cyclic polymers of monomeric silicon/oxygen monomers, in which a polymeric backbone is made up of alternating silicon and oxygen atoms. The silicon atoms may carry a wide variety of substituents, which can be the same or different.

“Film-forming” ingredients are chemicals that produce a continuous film on skin.

Compositions of the present invention preferably have a water resistance of 80 minutes and are comprised of at least one, preferably at least two, film-forming polysiloxane polymers.

In particularly preferred embodiments, the two film-forming polymers are (i) bis-vinyl dimethicone/dimethicone copolymer, commercially available in combination with dimethicone or cetyl dimethicone and (ii) dimethicone (and) acrylates/dimethicone copolymer.

Bis-vinyl dimethicone/dimethicone copolymer is a copolymer of dimethicone end-blocked with vinyl dimethicone.

Dimethicone (C₂H₆OSi)ₓC₄H₁₂Si is a mixture of fully methylated linear siloxane polymers end blocked with trimethylsiloxy units; it conforms generally to the formula:

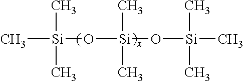

Vinyl dimethicone is a derivative of dimethicone in which some of the methyl groups are replaced with vinyl groups. The vinyl groups can occur at the ends of the siloxane chain may be pendant to the siloxane chain. It conforms generally to the formula:

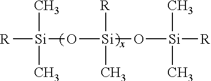

where R is a methyl or vinyl group, and at least one vinyl group is present.

Bis-vinyl dimethicone is a derivative of dimethicone in which one methyl group at each end of the siloxane chain is replaced with a vinyl group. It conforms generally to the formula:

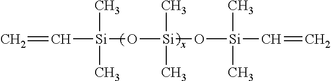

Cetyl dimethicone is a dimethyl siloxane polymer that conforms to the formula:

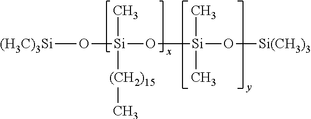

Acrylates/dimethicone copolymer is a copolymer of dimethicone and one or more monomers of acrylic acid, methacrylic acid or one of their simple esters.

In addition to the two dimethicone copolymer film formers (discussed immediately above), the broad-spectrum photoprotective compositions of the invention are comprised of at least two, preferably three silicone compounds selected from methicone, dimethicone, simethicone, cetyl dimethicone and phenyl trimethicone.

More preferably, a first silicone compound is a silicone wax (preferably cetyl dimethicone), and a second silicone compound is a silicone fluid (preferably selected from the group of methicone, dimethicone, simethicone, and phenyl trimethicone).

Methicone is a linear monomethyl polysiloxane that conforms generally to the formula:

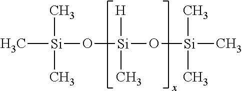

Simethicone is a mixture of dimethicone with an average chain length of 200 to 350 dimethylsiloxane units and silica.

Phenyl trimethicone is the siloxane polymer that conforms generally to the following formula, in which x is predominantly 1 to 3:

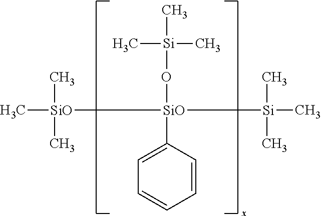

Phenyl trimethicone is preferably used in combination with (a) disteardimonium hectorite and (b) triethyl citrate. Disteardimonium hectorite is the reaction product (a) hectorite, one of the montmorillonite minerals that are the principal constituents of bentonite clay and (b) distearyldimonium chloride, a quaternary ammonium salt. Triethyl citrate is a triester of ethyl alcohol and citric acid. The above combination is commercially available as Bentone Gel PTM V from Elementis Specialities, Inc. (Hightstown, NJ) and functions as a non-surfactant dispersing agent, preventing syneresis and settling of photoprotective metal oxide particles.

In certain preferred embodiments, at least two dimethicones are included in the broad-spectrum photoprotective compositions of the invention: a first dimethicone having a lower kinematic viscosity in the range of 0.65 to 400 centistokes (cst), preferably about 50 cst; and a second dimethicone having a higher kinematic viscosity in the range of 1,000 to 5,000 cst.

Dimethicone and cetyl dimethicone are preferably present in broad-spectrum photoprotective compositions of the invention at a concentration of from 1 about 5%; preferably less than about 4.5, still more preferably about 4.0.

### Additional Ingredients

Broad-spectrum photoprotective compositions of the present invention preferably include one or several ingredients that (a) reduce visible redness (i.e., erythema) or inflammation (known in the art as anti-inflammatory) or act as an antioxidant (i.e., reduce oxidative damage; also known in the art as free radical quenchers), (b) reduce the appearance of the signs of skin aging, which can include fine lines, wrinkles, uneven pigmentation (dyschromia), loss of elasticity or firmness, increased dryness, reduced skin moisture, loss of softness/suppleness (collectively “anti-aging ingredients”), or (c) oil-absorbent powders. Non-limiting examples of antioxidants and anti-aging ingredients include: vitamins and derivatives thereof, preferably, ascorbic acid (vitamin C) and its salts; ascorbyl esters of fatty acids, ascorbic acid derivatives (e.g., magnesium ascorbyl phosphate, sodium ascorbyl phosphate, ascorbyl sorbate); tocopherol (vitamin E), tocopherol sorbate, tocopherol acetate, other esters of tocopherol; coenzyme Q10 (ubiquinone, ubiquinol) and its analogues, including without limitation, idebenone; bioflavonoids; amino acids; peptides, preferably comprised of two to ten amino acids, still more preferably lipidated peptides; superoxide dismutase; dipotassium glycyrrhizinate; tea extract or coffee extract; stem cells, including stem cell lysates; ceramides; as well as ingredients known in the art as humectants, moisturizers, skin-conditioning agents, skin soothing and/or healing agents.

Non-limiting examples of antioxidants/radical scavengers/moisturizing agents which may be topically delivered in the broad-spectrum photoprotective compositions of the present invention include: bisabolol; Camellia sinensis leaf extract; Capparis spinosa fruit extract; hydroxyacetophenone; Olea europaea (olive) fruit extract, preferably containing hydroxytyrosol (10% HPLC); Opuntia ficus-indica extract; Polygonum aviculare extract; Zingiber officinale root extract.

Preferred, but non-limiting examples of oil-absorbent powders include nylon-12 and polymethylsilsesquioxane.

Nylon-12 is a polyamide derived from 12-aminododecanoic acid and conforms generally to the formula:

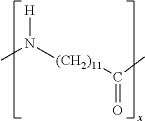

Polymethylsilsesquioxane is a polymer formed by the hydrolysis and condensation of methyltrimethoxysilane.

In certain embodiments, nylon-12 is combined with polymethyl methacrylate or polymethyl-silsesquioxane.

Certain embodiments of the present invention include ingredients that act as pollutant scavengers (i.e., entrap or reduce the negative effects of environmental particulate matter on the skin). By “negative effects on the skin” is meant collagen degradation, overproduction of melanin, and inflammation. Non-limiting examples of preferred pollutant scavengers include Plankton Glass® (described above) and benzylidene dimethoxydimethylindanone (available under the tradename SymUrban® from Symrise, Teterboro, NJ).

Compositions of the present invention preferably contain one or several ingredient(s) that absorb, attenuate or reduce negative effects on the skin caused by blue light and/or infrared radiation.

One non-limiting, preferred example of an ingredient that counters the effects of infrared radiation on the skin is a combination of PMMA, cerium oxide, aluminum oxide, available from Argan under the tradename ARG-NIR.

One non-limiting, preferred example of an ingredient that counters the effects of blue radiation on the skin is lutein, available as FloraGLO™ Lutein 5% Topical [Carthamus tinctorius (safflower) seed oil (and) Tagetes erecta flower extract] and FloraGLO™ Lutein 10% Topical [Water-dispersible granules of sucrose (and) tapioca starch (and) xanthophyll].

Sunless tanning agents, including but not limited to the alpha-MSH biomimetic peptide acetyl hexapeptide-1, may be added to the broad-spectrum photoprotective compositions of the present invention.

In certain preferred embodiments, broad-spectrum sunscreen compositions of the present invention contain an ingredient that reduces pigmentation induced by both infrared light and visible light (from 400-700 nm), preferably carnosine.

Carnosine (available from Symrise under the tradename Dragosine®) is heterocyclic amine that conforms to the formula:

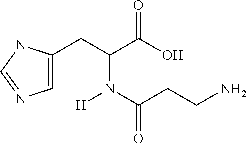 [SMILES: NCCC(=O)NC(Cc1cnc[nH]1)C(=O)O]

In certain preferred embodiments, carnosine is present at a concentration of from 0.01-0.2%.

Compositions of the present invention preferably do not contain any of the following: paraben, formaldehyde, chlorphenesin, and phenoxyethanol. Instead, as a preferred preservative system, compositions of the present invention have a synergistic combination of: hydroxyacetophenone (preferably at 0.5%); a mixture of 1,2 hexanediol (and) caprylyl glycol (preferably at a combined concentration of 0.5%); a mixture of bisabolol (and) Zingiber officinale (ginger) extract (preferably at a combined concentration of 0.1%). This synergistic combination passes the microbial enumeration test found in Chapter 61 of the US Pharmacopeia.

The invention is further defined by reference to the following examples. These examples are representative and should not be construed to limit the scope of the invention.

| Phase | Ingredient(s) | SPF 30 | SPF 55 |
|---|---|---|---|
| A | Water | Q.S. | Q.S. |
| A | Glycerin 99.7%, USP | 0.25-1.00 | 0.25-1.00 |
| A | Panthenol | 0.10-0.50 | 0.10-0.50 |
| A | Allantoin | 0.05-0.25 | 0.05-0.25 |
| A | 1,2 Hexanediol and Caprylyl Glycol | 0.10-1.00 | 0.10-1.00 |
| A | Hydroxyacetophenone | 0.10-1.00 | 0.10-1.00 |
| A | Sodium Chloride | 0.25-1.50 | 0.25-1.50 |
| A | Niacinamide | 0.10-3.00 | 0.10-3.00 |
| B | Butyl Octyl Salicylate | 15.0 | 12.5 |
| B | Squalane | 0.50-2.00 | 0.50-2.00 |
| B | Cetyl Dimethicone (and) Bis-Vinyl | 4.0 | 4.0 |
| | Dimethicone/Dimethicone Copolymer | | |
| B | Dimethicone (and) Bis-Vinyl | 4.0 | 4.0 |
| | Dimethicone/Dimethicone Copolymer | | |
| B | Styrene/Acrylates Copolymer (2.00-5.50) | 2.5 | 5.5 |
| B | Polyhydroxystearic Acid | 0.25-1.00 | 0.25-1.00 |
| B | Zinc Oxide | 5.0 | 10.00 |
| B | Iron Oxides | 1.5-2.0 | 0.0 |
| C | Phenyl Trimethicone, Disteardimonium | 1.50-3.00 | 1.50-3.00 |
| | Hectorite, (and) Triethyl Citrate | | |
| C | Caprylic/Capric Triglyceride, Titanium | 3.5 | 3.5 |
| | Dioxide, Aluminum Hydroxide, Stearic | | |
| | Acid (and) Polyhydroxystearic Acid | | |
| C | Dimethicone (and) Acrylates/Dimethicone | 1.00-3.00 | 1.00-3.00 |
| | Copolymer | | |

Preferably, iron oxides are coated. Preferred, but non-limiting, examples of coated iron oxides are iron oxide, CI 77491; iron oxide, CI 77499 and iron oxide, CI 77492, each surface treated/coated with hydrogenated lecithin.

The following ingredients may be added to the above formulations to the indicated Phase in the indicated concentration range:

| Phase | Ingredient(s} | % wt/wt |
|---|---|---|
| A | Capparis Spinosa Fruit Extract, Opuntia Ficus-indica | 0.10-3.0 |
| | Extract, Olea Europea (Olive) Leaf Extract and Starch | |
| A | Olea Europea (Olive) Fruit Extract and Starch | 0.10-3.0 |
| A | Carosine | 0.01-0.2 |
| C | Bisabolol and Zingiber Officinale (Ginger) Root Extract | 0.01-0.1 |
| C | Tocopheryl Acetate | 0.01-3.0 |
| E | PMMA, Cerium Oxide, Aluminum Oxide | 0.10-2.0 |
| E | Ubiquinone, Tocopheryl Acetate, C₁₂₋₁₅ Alkyl Benzoate | 0.25-2.0 |
| E | Water, Glycerin, Polygonum Aviculare Extract | 0.50-2.0 |
| E | Water, Glycerin, Camellia Oleifera Leaf Extract | 0.25-3.0 |

Combine Phase A ingredients. Heat and mix at 65-70° C. In a separate vessel, combine Phase B ingredients. Heat and mix at 40-50° C. Mix until ZnO is uniformly dispersed. Homogenize for approximately 8-10 minutes, until ZnO is coated with cetyl dimethicone and dimethicone.

Combine Phase C ingredients with Phase B ingredient. Mix until TiO₂ is uniform dispersed. Homogenize for approximately 1 minute. At 65-70° C., while mixing with high speed, very slowly add Phase A to the oil phase. Mix for 5-10 minutes. Homogenize for 1 minute. Add Phase D ingredient after homogenization; mix until uniformly dispersed. Continue mixing and cool mixture of A+B+C+D. When cooled to 50° C., add Phase E ingredients; mix until homogeneous. Cool final mixture A+B+C+D+E to 30° C.

The synergistic combination of ZnO and TiO₂ with (a) two SPF boosters—butyl octyl salicylate (BOS) and Sunspheres®, and (b) two siliceous compounds—an amorphous spherical silica and a mixture of diatomaceous algal species, each having photonic and porous silica crystals (Plankton Glass Flower®, “PGF”), in the ratios described above, achieve a critical wavelength (“CW”) of at least 370 nm necessary to qualify as a “broad-spectrum” (UVA/UVB) sunscreen under applicable FDA regulations (as of the filing date of the present application). Combining these ingredients in ratios other than as described above do not achieve a critical wavelength of at least 370 nm.

| Prototype | CW (nm) | BOS | Sunspheres ® | ZnO | TiO2 | Silica | PGF |
|---|---|---|---|---|---|---|---|
| 1 | 368.13 | 13.00% | 2.50% | 10.00% | 3.50% | 2.50% | 0.50% |
| 2 | 371.80 | 13.00% | 5.50% | 10.00% | 3.50% | 2.00% | 0.50% |
| 3 | 375.88 | 13.00% | 5.50% | 10.00% | 3.50% | 2.50% | 0.50% |

While the illustrative embodiments of the invention have been described with particularity, it will be understood that various other modifications will be apparent to and can be readily made by those skilled in the art, without departing from the spirit and scope of the invention. Accordingly, it is not intended that the scope of the claims appended hereto be limited to the examples and descriptions set forth hereinabove but rather that the claims be construed as encompassing all the features of patentable novelty in the present invention, including all features which would be treated as equivalents by persons having ordinary skill in the art of formulating topically-applied personal care and dermatological products.

What is claimed is:

1. A broad-spectrum, stable, mineral sunscreen composition, in the form of a water-in-oil emulsion, prepared by the process comprising:
(1) preparing a first mixture comprising (a) porous zinc oxide particles having an average particle size of greater than 100 nanometers, (b) polyhydroxystearic acid, (c) squalane, and (d) a formulation component comprising a first film-forming ingredient comprising bis-vinyl dimethicone/dimethicone copolymer in a solvent comprising either (I) cetyl dimethicone, (II) dimethicone, or (III) both cetyl dimethicone and dimethicone;
(2) mixing the first mixture such that the porous zinc oxide particles are uniformly dispersed in the first mixture and the porous zinc oxide particles are infiltrated by the formulation component;
(3) homogenizing the first mixture;
(4) preparing a second mixture comprising (a) titanium dioxide particles, caprylic/capric triglyceride, aluminum hydroxide, stearic acid, and polyhydroxystearic acid, (b) a suspending dispersant component comprising phenyl trimethicone, disteardimonium hectorite, and triethyl citrate, and (c) a second film-forming ingredient comprising acrylates/dimethicone copolymer in dimethicone;
(5) combining the first mixture and the second mixture to form a third mixture, the third mixture further comprising at least one polyglycerin emulsifier containing 3-6 glycerin units;
(6) mixing the third mixture until the titanium dioxide particles are uniformly dispersed in the third mixture;
(7) preparing a fourth mixture comprising water, glycerin, and sodium chloride;
(8) adding the fourth mixture to the third mixture while mixing the third mixture to form an emulsion, and thereafter homogenizing the emulsion to form a fifth mixture; and
(9) adding two siliceous compounds to the fifth mixture to form the broad-spectrum, stable, mineral sunscreen composition, wherein the two siliceous compounds consist of amorphous spherical silica and a mixture of materials from different diatomaceous algal species, wherein each of the algal species comprises photonic and porous silica crystals, wherein the process is performed in a manner such that (i) the zinc oxide and titanium dioxide particles are uniformly dispersed in the composition, (ii) the composition does not separate into oil and water phases or experience particle agglomeration or particle settling after storage at (I) 50° C. for one month or (II) 40° C. for three months, (iii) the composition does not cause visible whitening when applied on the skin of a person having a Fitzpatrick Skin Type of I-IV, and (iv) the composition provides a sun protection factor (SPF) of at least 50.

2. The composition of claim 1, wherein the composition does not comprise polyethylene glycol.

3. The composition of claim 2, wherein the first mixture further comprises (a) butyloctyl salicylate, (b) styrene/acrylates copolymer, or (c) both butyloctyl salicylate and styrene/acrylates copolymer.

4. The composition of claim 3, wherein the composition further comprises poly(methyl methacrylate).

5. The composition of claim 2, wherein the titanium dioxide particles comprise about 3.5 wt. % of the composition.

6. The composition of claim 5, wherein a calculated ratio of the amorphous spherical silica to the mixture of materials of the different diatomaceous algal species is about 1:4 wt./wt. to about 50:1 wt./wt.

7. The composition of claim 6, wherein the mixture of diatomaceous algal species comprises at least two of Fragilaria zeilleri, Gomphonema angustatum, Navicula radiosa, and Cyclotella andancensis.

8. The composition of claim 7, wherein the at least one polyglycerin emulsifier has a hydrophobic lipophilic balance of about 3 to about 8.

9. The composition of claim 8, wherein the squalane makes up about 0.5 wt. % to about 2 wt. % of the composition.

10. The composition of claim 9, wherein the composition further comprises (a) aluminum oxide particles, (b) cerium oxide particles, or (c) both aluminum oxide particles and cerium oxide particles.

11. The composition of claim 1, wherein the composition further comprises iron oxide particles in an amount sufficient to impart a tint to the composition.

12. The composition of claim 1, wherein the composition is water resistant for at least 80 minutes according to the test method of Section 352.76(b) of Title 21 of the United States Code of Federal Regulations in effect on Mar. 23, 2018.

13. The composition of claim 2, wherein the composition does not comprise (a) poly(methyl methacrylate) (b) styrene/acrylates copolymer, (c) butyloctyl salicylate, or (d) any combination of (a)-(c).

14. A broad-spectrum, stable, mineral sunscreen composition, in the form of a water-in-oil emulsion, prepared by the process comprising:
(1) preparing a first mixture comprising (a) porous zinc oxide particles having an average particle size of greater than 100 nanometers, (b) a first film-forming ingredient comprising bis-vinyl dimethicone/dimethicone copolymer in a solvent comprising either (I) cetyl dimethicone, (II) dimethicone, or (III) both cetyl dimethicone and dimethicone,
(2) mixing the first mixture such that (a) the porous zinc oxide particles are coated with the first film-forming ingredient, and (b) the porous zinc oxide particles are uniformly dispersed in the first mixture, and (c) the porous zinc oxide particles are infiltrated by the first film-forming ingredient in a manner that significantly increases transparency of the porous zinc oxide particles;
(3) preparing a second mixture comprising (a) titanium dioxide particles, caprylic/capric triglyceride, aluminum hydroxide, stearic acid, and polyhydroxystearic acid, and (b) a second film-forming ingredient comprising acrylates/dimethicone copolymer in a dimethicone solvent;
(4) combining the first mixture and the second mixture to form a third mixture and mixing the third mixture until the titanium dioxide particles are uniformly dispersed in the third mixture, the third mixture further comprising at least one polyglycerin emulsifier containing 3-6 glycerin units and having a hydrophobic lipophilic balance of about 3 to about 8;
(5) preparing the broad-spectrum, stable water-in-oil emulsion, mineral sunscreen composition by adding water to the third mixture while mixing the third mixture, wherein (a) the composition does not comprise polyethylene glycol, (b) the composition does not separate into oil and water phases and does not experience particle agglomeration or particle settling after storage at (I) 50° C. for one month or (II) 40° C. for three months, (c) the composition does not cause visible whitening when applied on the skin of a person having a Fitzpatrick Skin Type of I-IV, (d) the composition provides a sun protection factor (SPF) of at least 50, (e) wherein the porous zinc oxide particles are present in an amount representing about 10 wt. % of the composition, (f) wherein the titanium dioxide particles are present in an amount representing about 3.5 wt. % of the composition, and (g) wherein the zinc oxide particles and the titanium dioxide particles are uniformly dispersed in the composition.

15. The composition of claim 14, wherein the composition is water resistant for at least 80 minutes according to the test method of Section 352.76(b) of Title 21 of the United States Code of Federal Regulations in effect on Mar. 23, 2018.

16. The composition of claim 15, wherein the composition does not comprise (a) butyl octyl salicylate, (b) a styrene/acrylates copolymer, (c) poly(methyl methacrylate), or (d) any combination of (a)-(c).
